(19) United States
(12) Patent Application Publication
(10) Pub. No.: US 2009/0104298 A1
(43) Pub. Date: Apr. 23, 2009

(54) USE OF ROOIBOS EXTRACT FOR PROTECTING HAIR COLOUR

(75) Inventors: Holger Joppe, Dassel (DE); Martina Herrmann, Hameln (DE); Jorn Wiedemann, Holzminden (DE)

Correspondence Address: CONNOLLY BOVE LODGE & HUTZ LLP, 1875 EYE STREET, N.W., SUITE 1100, WASHINGTON, DC, 20006, US

(73) Assignee: SYMRISE GmbH & Co. KG, Holzminden (DE)

(21) Appl. No.: 12/094,347

(22) PCT Filed: Nov. 6, 2006
(86) PCT No.: PCT/EP2006/068092

§ 371 Date: Sep. 30, 2008

Related U.S. Application Data
(60) Provisional application No. 60/738,054, filed on Nov. 21, 2005.

(51) Int. Cl. A61K 36/48; A61Q 5/00

(52) U.S. Cl. 424/774; 424/779

(57) ABSTRACT

The invention relates to the use of an extract of fermented and/or unfermented rooibos leaves and/or stems for reducing or slowing the loss of the natural or artificial colour of hair.

The present invention relates to the use of rooibos extracts, in particular in cosmetic agents for hair cleansing, hair care, hair dyeing and hair styling, for reducing or slowing colour loss in dyed and undyed hair as a result of, for example, sunlight or treatment with a hair-cosmetic preparation.

Human hair is exposed to many harmful influences. As well as leading to structural damage, such influences can also have more or less clearly visible effects. For example, normal exposure to sunlight, that is to say exposure that is not at all excessive, can cause such considerable damage to the hair that its attractiveness is visibly reduced.

The most highly visible damage naturally includes all hair damage that is accompanied by a colour change, and natural hair colour can fade just as much as artificially applied hair colours. The dyeing of keratin-containing fibres, such as, for example, hair, is generally carried out either using direct dyes or using oxidation dyes, which are formed by the oxidative coupling of one or more developer components with one another or with one or more coupler components. Coupler and developer components are also referred to as oxidation dye precursors. The dyeing can be temporary, semi-permanent or permanent in nature.

Colour changes as a result of colour loss do not normally occur uniformly, so that an unattractive, patchy overall appearance of the hair generally results. In the worst case, synthetic hair colours produce not a constant lightening effect but an alteration in the colour shade owing to the different light sensitivities of the individual colour-giving components.

In addition to exposure to sunlight and artificial UV light, for example in the solarium, hair washing and permanent wave treatment, for example, also lead to more rapid fading of the hair colour, in particular of dyed hair.

It was an object of the present invention, therefore, to provide agents for reducing or slowing colour change, that is to say the loss of the natural and/or artificial hair colour. In particular, agents are to be provided for reducing or slowing the change in hair colour as a result of exposure to light (e.g. sunlight and/or artificial UV radiation (e.g. in the solarium)) and/or as a result of treatment with hair-cosmetic preparations (e.g. in the case of hair washing and/or permanent wave treatment).

Although active ingredients for reducing hair colour loss are already in use in the mentioned technical fields, the search for alternatives continues. It would be desirable in this connection to have available a product that is able to develop its hair-protecting action both before and after exposure to, for example, sunlight or a hair-cosmetic preparation. When seeking alternative agents it must be ensured, however, that the substances used are toxicologically harmless, are well tolerated by the skin and hair, are inexpensive to produce and are stable (in particular in conventional cosmetic formulations) and that their own individual odour is as weak as possible. In accordance with the continuing trend towards natural active ingredients, novel active ingredients of natural, in particular plant, origin are particularly desirable.

Attempts have already been made, with comparatively little success, to protect hair with UV absorbers such as are used very successfully in skin cosmetics. However, such attempts usually fail because only very small layer thicknesses can be applied to the hair before the appearance becomes adversely affected. For example, when conventional UV absorbers are used on grey hair, it frequently appears yellow owing to its own inherent colour. Light-scattering pigments based on titanium dioxide, on the other hand, are all too visible particularly on dark hair. In addition, it is not as simple on hair to produce a cohesive protective film without undesirable effects, such as a greasy appearance and a sticky feel to the hair. Furthermore, unlike the development of sunburn on the skin, hair colour is damaged not only by UV light but also by the entire wavelength range of the visible spectrum. In order to provide protection against visible light, it would accordingly be necessary to apply dark or highly light-scattering (and hence colour-changing) protective substances to the hair.

WO 2004/071473 describes the use of phenolic diterpenes, such as carnosolic acid, carnosol and derivatives thereof, for protecting hair colour. However, these substances are very complex and expensive to prepare in pure form and, in addition, they are not very stable in pure or highly concentrated (e.g. from rosemary or sage extracts) form.

Offenlegungsschrift WO 2004/082646 reports on the use of polyphenols or plant extracts containing them, in particular grape skin, purple carrot, tea or cocoa extract, for reducing the colour loss of dyed hair.

Offenlegungsschrift EP 1 348 419 reports on the use of pomegranate extracts to maintain the hair colour and colour intensity of dyed hair. The effect is attributed to the tannins, such as ellagitannins, contained therein.

Compared with the extracts used in WO 2004/082646 and EP 1 348 419, rooibos extracts have a lower polyphenol content (tannins are polyphenols) and nevertheless exhibit better activity (see in this connection also Example 13 hereinbelow).

Rooibos or red bush (Aspalathus linearis) is originally an endemic species of the South African Cape province and belongs to the family of the Papilionaceae (Fabaceae). The plant is nowadays cultivated on a large scale in South Africa for tea production and is marketed globally. In order to produce tea, the leaves and branch tips are harvested, cut, crushed, fermented and dried. Recently, as well as fermented rooibos tea, unfermented “green” rooibos has also increasingly been found on the market.

Unlike Camellia sinensis tea (e.g. black, green or white tea), rooibos is distinguished by the absence of caffeine and a low content of polyphenols and, in particular, tannins. The following have hitherto been described as constituents: flavonoids, inter alia C-glycosyl flavones such as orientin, isoorientin, vitexin and isovitexin, flavonol glycosides such as isoquercitrin, hyperoside, rutin, the dihydrochalcones aspalathin and nothofagin, and eriodicytol derivatives (L. Bramati et al., J. Agric. Food Chem. 2002, 50, 5513-5519; C. Marais et al., Phytochem. 2000, 55, 43-49).

Fermented and “green” (unfermented) rooibos are markedly different in their aspalathin contents. While unfermented rooibos tea contains from 3 to 12 g of aspalathin/100 g, the content falls to 0.2 to 1.3 g/100 g as a result of fermentation (H. Schulz et al., Deutsche Apotheker Zeitung 2000, 140, 3809-3815).

Rooibos possesses various biological activities. For example, its anti-inflammatory, antiallergenic, antimutagenic, antimicrobial, antiviral, hepatoprotective and antioxidative activity has been described in the literature, the antioxidative action of unfermented rooibos being described as markedly better than that of fermented rooibos. In Euro Cosmetics 2003 (9), 20-23, F. Otto et al. describe that approximately 70% of the original antioxidative action of “green” rooibos is lost on fermentation. Up to 90% of the aspalathin is lost on fermentation.

Improvements in hair growth and in the condition of the hair as a result of rooibos extract have also been reported (J. Tiedtke et al., Euro Cosmetics 2002 (6), 16-19).

However, an effect of rooibos extracts in reducing the loss of natural and/or artificial hair colour induced, for example, by sunlight or by hair cosmetics has not hitherto been described.

Surprisingly, our own extensive studies have shown that extracts of rooibos (Aspalathus linearis) exhibit outstanding activity in protecting hair colour, in particular against treatment with light and/or hair-cosmetic formulations. It was also surprising that extracts of both fermented and “green”, unfermented rooibos were highly effective. In the tests which have been carried out, fermented and unfermented rooibos extracts exhibited comparable activity, that is to say the colour-protecting activity is not, or not only, based on the antioxidative action of rooibos extracts.

The invention accordingly relates primarily to the use of an extract of fermented and/or unfermented rooibos leaves and/or stems for reducing or slowing the loss of the natural or artificial colour of hair.

The invention additionally relates to a method of reducing or slowing the light-induced loss of the natural or artificial colour of hair, comprising the following step:
application of an extract of fermented and/or unfermented rooibos leaves and/or stems to the hair.

The rooibos extract used for reducing or slowing the change in hair colour can be extracts of fermented or unfermented rooibos leaves and stems prepared by extraction with water, methanol, ethanol, isopropanol or other solvents or solvent mixtures of comparable polarity. Particularly good results are obtained by the use of extracts of fermented rooibos leaves and stems, in particular aqueous extracts.

The rooibos extract used can be in the form of a solid, in pure form or with the addition of a cosmetically acceptable solid carrier, such as, for example, Neo-Extrapon Bio-Rooibos (obtainable from Symrise) used in Example 10. However, it can also be in the form of a liquid, that is to say with the addition of a cosmetically acceptable solvent, such as, for example, glycerol, 1,2-propylene glycol, 1,3-butylene glycol, ethanol, water and mixtures of two or more of the mentioned solvents with water (optionally with the addition of a solubiliser, preservative or antioxidant), such as, for example, Extrapon Rooibos GW (also obtainable from Symrise) used in Examples 4 and 6.

Rooibos extract can advantageously be used in the cleansing, care, dyeing and styling of hair wherever cosmetically desirable effects are associated with a reduction in or slowing of the colour loss of natural and/or dyed hair. To that end, it is preferably applied topically to the hair to be treated, preferably in the form of a hair-cosmetic preparation.

The invention accordingly relates also to a method of reducing the hair-bleaching action or increasing the hair-colour-stabilising action of a hair-cosmetic fomulation, wherein (a) an extract of fermented and/or unfermented rooibos leaves and/or stems is added to the preparation and/or (b) the extract of fermented and/or unfermented rooibos leaves and/or stems is applied to the hair before, during or after application of the preparation to the hair. The statements made hereinbefore apply in respect of preferred rooibos extracts.

Important fields of application of the rooibos extract to be used according to the invention are hair-cosmetic preparations which (apart from the presence of rooibos extract) have the conventional composition and are used for the cosmetic treatment, cleansing, care, dyeing and styling of the hair. Accordingly, such preparations can be in the form of, for example, cleansing agents such as, for example, shampoo (including shampoo for normal hair, for hair that quickly becomes greasy, for dry, mistreated (damaged) hair, for dyed hair, 2-in-1 shampoo, anti-dandruff shampoo, shampoo for dry scalp, shampoo concentrate), soap, syndet, liquid washing, shower and bath preparation, care preparation such as, for example, emulsion (in the form of a solution, dispersion, suspension; cream, lotion or milk, depending on the preparation process and ingredients of the type W/O, O/W or multiple emulsion, PIT emulsion, emulsion foam, microemulsion, nanoemulsion, Pickering emulsion), ointment, paste, gel (including hydrogel, hydrodispersion gel, oleogel), alcoholic or aqueous/alcoholic solution, oil, toner, balm, serum, powder, wipe, eau de toilette, eau de Cologne, perfume, wax, including the form of application as a (pump) spray, aerosol (foaming, non-foaming or post-foaming), as an insect repellent, as a sun protection agent, as an after-sun preparation, as a care preparation such as, for example, conditioner, hair treatment, hair lotion, hair rinse, hair cream, pomade, permanent wave agent and fixative, hair smoothing agent (anti-frizz agent, relaxer), setting lotion, styling aid (e.g. spray, gel or wax), hair dye such as, for example, direct and/or oxidation hair dyes, temporary, semi-permanent and/or permanent hair dyes.

For use in applications or methods according to the invention, the concentration of rooibos extract (in solid or liquid form) in hair-cosmetic preparations is preferably in the range from 0.0001 to 20 wt. %, more preferably in the range from 0.0001 to 10 wt. % and particularly preferably in the range from 0.001 to 5 wt. %, based on the total weight of the preparation.

The hair-cosmetic formulations according to the invention can contain cosmetic auxiliary substances and additives as are conventionally employed in such preparations, for example further hair-colour-protecting active ingredients, sun protection agents, preservatives, bactericides, fungicides, virucides, cooling active ingredients, insect repellents (e.g. DEET, IR 3225), plant extracts, plant parts, anti-inflammatory active ingredients, anti-itching active ingredients, substances that accelerate the healing of wounds (e.g. chitin or chitosan and derivatives thereof), film-forming substances (e.g. polyvinylpyrrolidones or chitosan or derivatives thereof), antioxidants, vitamins, 2-hydroxycarboxylic acids (e.g. citric acid, malic acid, L-, D- or dl-lactic acid), proteins, hydrolysed proteins, active ingredients for promoting hair growth or inhibiting hair growth, caring agents (e.g. cholesterol, ceramides, pseudoceramides), softening, moisturising and/or humectant substances, fats, oils, saturated fatty acids, mono- or poly-unsaturated fatty acids, α-hydroxy acids, polyhydroxy fatty acids or derivatives thereof, waxes or other conventional constituents of a hair-cosmetic formulation, such as alcohols, polyols, polymers, foam stabilisers, electrolytes, osmolytes, organic solvents, silicone derivatives or chelating agents (e.g. ethylenediamine-tetraacetic acid and derivatives), anti-dandruff active ingredients (e.g. climbazole, ketoconazole, piroctonoleamine, zinc pyrithione), hair-care agents, perfumes, substances for preventing foaming, colourings, pigments that have a colouring action, thickeners (advantageously silicon dioxide, aluminium silicates, such as, for example, bentonites, polysaccharides or derivatives thereof, e.g. hyaluronic acid, guar flour, xanthan gum, hydroxypropylmethylcellulose or allulose derivatives, particularly advantageously polyacrylates such as, for example, carbopols or polyurethanes), surface-active substances and emulsifiers.

The amounts of hair-cosmetic auxiliary substances and additives and of perfume to be used in a particular case can readily be determined by the person skilled in the art, in dependence on the nature of the product in question, by means of simple testing.

Preferred embodiments and further aspects of the present invention will become apparent from the accompanying patent claims and from the following examples, which are not intended to limit the invention:

### EXAMPLES 1-9

### Formulations for the Use According to the Invention of Rooibos Extract Having Hair-Colour-Protecting Action

In Table 1 below

1=colour-protecting shampoo

2=2-in-1 after-sun shampoo for coloured hair

3=colour-protecting after-sun hair conditioner, leave-in

4=hair conditioner, rinse off

5=hair-styling gel for coloured hair

6=pump hairspray with UV protection

7=hair-end fluid, without silicone oil

8=colour-protecting anti-dandruff shampoo

9=colour shampoo (pH 7.0)

TABLE 1

| RAW MATERIAL | | | | | | | | | | |
|---|---|---|---|---|---|---|---|---|---|---|
| NAME | | WEIGHT % | | | | | | | | |
| (SUPPLIER) | INCI | 1 | 2 | 3 | 4 | 5 | 6 | 7 | 8 | 9 |
| Rooibos Extract | Aspalathus Linearis | 0.1 | | 0.02 | | | | 0.005 | 0.05 | |
| | Leaf Extract | | | | | | | | | |
| Neo-Extrapone | Maltodextrin, | | 0.5 | | | 0.2 | | | | 1.0 |
| Organic Rooibos | Aspalathus Linearis | | | | | | | | | |
| (Symrise) | Leaf Extract | | | | | | | | | |
| Extrapone | Water (Aqua), | | | | 2.0 | | 0.3 | | | |
| Rooibos GW | Glycerin, | | | | | | | | | |
| (Symrise) | Aspalathus Linearis | | | | | | | | | |
| | Leaf Extract | | | | | | | | | |
| Aloe Vera Gel | Water (Aqua), Aloe | 1.0 | | | 0.5 | | | | | |
| Concentrate 10/1 | Barbadensis Leaf | | | | | | | | | |
| (Symrise) | Juice | | | | | | | | | |
| -(-Alpha-)- | Bisabolol | | | | | | | | 0.1 | |
| Bisabolol, natural | | | | | | | | | | |
| (Symrise) | | | | | | | | | | |
| AMP-95 (Angus) | Aminomethyl- | | | | | | 0.7 | | | |
| | propanol | | | | | | | | | |
| Antil 141 Liquid | Propylene Glycol, | | 1.0 | | | | | | | |
| (Degussa- | PEG-55 Propylene | | | | | | | | | |
| Goldschmidt) | Glycol Distearate | | | | | | | | | |
| Antil 171 | PEG-18 Glyceryl | | | | | | | | 2.0 | |
| (Degussa- | Oleate/Cocoate | | | | | | | | | |
| Goldschmidt) | | | | | | | | | | |
| Antil 200 | PEG-200 | 1.5 | | | | | | | | |
| (Degussa- | Hydrogenated | | | | | | | | | |
| Goldschmidt) | Glyceryl Palmitate, | | | | | | | | | |
| | PEG-7 Glyceryl | | | | | | | | | |
| | Cocoate | | | | | | | | | |
| Carbopol ETD | Carbomer | | | | | 0.7 | | | | |
| 2001 (Noveon) | | | | | | | | | | |
| Citric Acid 10% | Citric Acid | | | | | | | 0.2 | | q.s. |
| sol. | | | | | | | | | | |
| Cremogen | Water (Aqua), | | | | 1.0 | | | | | |
| AlphaPulp | Butylene Glycol, | | | | | | | | | |
| (Symrise) | Malic Acid, Prunus | | | | | | | | | |
| | Amygdalus Dulcis | | | | | | | | | |
| | (Sweet Almond) | | | | | | | | | |
| | Seed Extract, | | | | | | | | | |
| | Actinidia Chinensis | | | | | | | | | |
| | (Kiwi) Fruit Juice, | | | | | | | | | |
| | Citrus Aurantium | | | | | | | | | |
| | Dulcis (Orange) | | | | | | | | | |
| | Juice, Citrus Para- | | | | | | | | | |
| | disi (Grapefruit) | | | | | | | | | |
| | Juice, Pyrus Malus | | | | | | | | | |
| | (Apple) Juice, PEG- | | | | | | | | | |
| | 40 Hydrogenated | | | | | | | | | |
| | Castor Oil | | | | | | | | | |
| Colour (Symrise) | Colour | | | | | | | | 0.2 | |
| Crinipan AD | Climbazole | | | | | | | | 0.5 | |
| (Symrise) | | | | | | | | | | |
| Dehyquart A CA | Cetrimonium | | 1.0 | 4.0 | | | | | | |
| (Cognis) | Chloride | | | | | | | | | |
| Dehyton K | Cocoamidopropyl | 6.0 | 8.0 | | | | | | | |
| (Cognis) | Betaine | | | | | | | | | |
| D-Panthenol 75L | Panthenol | | 1.0 | 1.0 | | | | 3.0 | | |
| (DSM Nutritional) | | | | | | | | | | |
| Dragocare W | PEG-40 Butyl- | | | | 1.0 | | | 3.0 | | |
| (Symrise) | octanol Wheat | | | | | | | | | |
| | Germ Esters, Water | | | | | | | | | |
| | (Aqua), Lactic Acid, | | | | | | | | | |
| | Tocopherol | | | | | | | | | |
| Dragocide Liquid | Phenoxyethanol, | 0.8 | 0.8 | 0.8 | 0.8 | 0.5 | | | 0.7 | 0.5 |
| (Symrise) | Methyl-, Ethyl-, | | | | | | | | | |
| | Butyl-, Propyl-, | | | | | | | | | |
| | Isobutylparaben | | | | | | | | | |
| Dragocolor Blue | Basic Blue 99 | | | | | | | | | 0.02 |
| (Symrise) | | | | | | | | | | |
| Dragocolor Brown | Basic Brown 17 | | | | | | | | | 0.1 |
| (Symrise) | | | | | | | | | | |
| Dragocolor | Basic Brown 16 | | | | | | | | | 0.1 |
| Mahogany | | | | | | | | | | |
| (Symrise) | | | | | | | | | | |
| Dragoderm | Glycerin, Triticum | 0.3 | 1.0 | 2.0 | 2.0 | | | | | |
| (Symrise) | Vulgare (Wheat) | | | | | | | | | |
| | Gluten, Water | | | | | | | | | |
| | (Aqua) | | | | | | | | | |
| Drago-Oat-Active | Water (Aqua), | | | 0.5 | | | | | | |
| (Symrise) | Butylene Glycol, | | | | | | | | | |
| | Avena Sativa (Oat) | | | | | | | | | |
| | Kernel Extract | | | | | | | | | |
| Ethanol 96% | Ethanol | | | | | 5.0 | 84.0 | 18.0 | | |
| Euperlan PK 771 | Glycol Distearate, | | | | | | | | 3.0 | |
| (Cognis) | Sodium Laureth | | | | | | | | | |
| | Sulfate, Cocamide | | | | | | | | | |
| | MEA, Laureth-10 | | | | | | | | | |
| Euperlan PK 900 | PEG-3 Distearate | | | | | | | | | 2.0 |
| BENZ-W (Cognis) | | | | | | | | | | |
| Euperlan PK 4000 | Glycol Distearate, | 1.0 | 2.5 | | | | | | | |
| (Cognis) | Laureth-4, Coco- | | | | | | | | | |
| | amidopropyl | | | | | | | | | |
| | Betaine | | | | | | | | | |
| Extrapone | Water (Aqua), | | | | | | | | 0.5 | |
| Camomile | Propylene Glycol, | | | | | | | | | |
| (Symrise) | Butylene Glycol, | | | | | | | | | |
| | Chamomilla | | | | | | | | | |
| | Recutita | | | | | | | | | |
| | (Matricaria) Flower | | | | | | | | | |
| | Extract, Glucose, | | | | | | | | | |
| | Bisabolol | | | | | | | | | |
| Extrapone | Water (Aqua), | | | 2.0 | | | | | | |
| Champagne GW | Glycerin, Wine | | | | | | | | | |
| (Symrise) | Extract, Alcohol | | | | | | | | | |
| Extrapone Green | Glycerin, Water | 0.1 | | | 0.2 | | | | | |
| Tea GW | (Aqua), Camellia | | | | | | | | | |
| (Symrise) | Sinensis Leaf | | | | | | | | | |
| | Extract | | | | | | | | | |
| Extrapone | Water (Aqua), | 0.5 | | | | | | | | |
| Passion Flower | Propylene Glycol, | | | | | | | | | |
| (Symrise) | Passiflora Incarnata | | | | | | | | | |
| | Extract, Glucose | | | | | | | | | |
| Extrapone | Glycerin, Water | | 0.3 | | | | | | 0.2 | |
| Rosemary GW | (Aqua), Rosmarinus | | | | | | | | | |
| (Symrise) | officinalis (Rose- | | | | | | | | | |
| | mary) Leaf Extract | | | | | | | | | |
| Frescolat ML | Menthyl Lactate | | | | | 0.8 | | | 0.5 | |
| (Symrise) | | | | | | | | | | |
| Genapol LRO | Sodium Laureth | 35.0 | | | | | | | | |
| liquid (Clariant) | Sulfate | | | | | | | | | |
| Genagen CAB | Cocamidopropyl | | | | | | | | | 2.0 |
| (Clariant) | Betaine | | | | | | | | | |
| Glycerin, 99.5% | Glycerin | | | | | 10.0 | | 8.0 | | |
| Hair Conditioner | Cetyl Alcohol, | | | | 3.7 | | | | | |
| Base (Symrise) | Behentrimonium | | | | | | | | | |
| | Chloride, Triticum | | | | | | | | | |
| | Vulgare (Wheat) | | | | | | | | | |
| | Bran Extract, | | | | | | | | | |
| | Linoleic Acid | | | | | | | | | |
| Lanette O | Cetearyl Alcohol | | | 3.5 | | | | | | |
| (Cognis) | | | | | | | | | | |
| Luviskol K 30 | PVP | | | | | 3.0 | | | | |
| Powder | | | | | | | | | | |
| (BASF AG) | | | | | | | | | | |
| Luviskol VA 37 E | PVP/VA Copolymer | | | | | | 3.0 | | | |
| (BASF AG) | | | | | | | | | | |
| Merquat 550 | Polyquaterinium-7 | 0.5 | 1.0 | | | | | | | |
| (Ondeo) | | | | | | | | | | |
| Mulsifan RT | C12-15 Pareth-12 | | | | | 4.0 | | | | |
| 203/80 (Z&S) | | | | | | | | | | |
| Natrosol 250HHR | Hydroxyethyl- | | | | | | | 0.6 | | |
| (Aqualon) | cellulose | | | | | | | | | |
| Neo Heliopan BB | Benzophenone-3 | 0.3 | 0.2 | | | | 0.2 | | 0.3 | |
| (Symrise) | | | | | | | | | | |
| Neo Heliopan | Phenylbenzimid- | | | | | | 0.2 | | | |
| Hydro (Symrise) | azole Sulfonic Acid | | | | | | | | | |
| Neo-PCL Water | Trideceth-9, PEG-5 | 1.0 | | | | | | | | |
| soluble N | Ethylhexanoate, | | | | | | | | | |
| (Symrise) | Water (Aqua) | | | | | | | | | |
| Neutrol TE | Tetrahydroxypropyl | | | | | 1.4 | | | | |
| (BASF) | Ethylenediamine | | | | | | | | | |
| PCL Liquid 100 | Cetearyl Octanoate | | | | | | 0.2 | | | |
| (Symrise) | | | | | | | | | | |
| Plantacare 1200 | Lauryl glucoside | | | | | | | | | 10.0 |
| UP (Cognis) | | | | | | | | | | |
| Polymer JR 400 | Polyquaternium-10 | | | | | | | | 0.2 | |
| (Nordmann, | | | | | | | | | | |
| Rassmann) | | | | | | | | | | |
| Polyquart H-81 | PEG-15 | | | 3.0 | | | | | | |
| (Cognis) | Cocopolyamine | | | | | | | | | |
| Pseudoceramide | N-(1-Hexa- | | 0.2 | 0.1 | | | | | | |
| 391 | decanoyl)-4- | | | | | | | | | |
| | hydroxy-L-prolin-(1- | | | | | | | | | |
| | hexadecyl-ester | | | | | | | | | |
| Rose CL forte | Water (Aqua), | | 0.5 | | | | | | | |
| (Symrise) | Glycerin, PEG-40 | | | | | | | | | |
| | Hydrogenated | | | | | | | | | |
| | Castor Oil, Rosa | | | | | | | | | |
| | Damascena Flower | | | | | | | | | |
| | Oil | | | | | | | | | |
| SF 1258 (Merck) | Cyclopentasiloxane, | | | | | | 0.1 | | | |
| | Cyclohexasiloxane | | | | | | | | | |
| Sodium Chloride | Sodium Chloride | 0.8 | 0.5 | 2.0 | | | | | 2.0 | |
| Sodium | Sodium Hydroxide | 0.5 | 0.1 | | 0.5 | | | | | |
| Hydroxide, 10% | | | | | | | | | | |
| sol. | | | | | | | | | | |
| Solubilizer | PEG-40 Hydro- | | | | | | | 2.0 | | |
| (Symrise) | genated Castor Oil, | | | | | | | | | |
| | Trideceth-9, | | | | | | | | | |
| | Propylene Glycol, | | | | | | | | | |
| | Water (Aqua) | | | | | | | | | |
| Symrise | Fragrance | 0.3 | 0.3 | 0.3 | 0.2 | 0.4 | 0.2 | 0.5 | 0.5 | 0.4 |
| Fragrance | | | | | | | | | | |
| Texapon K 14 S | Sodium Myreth | | | | | | | | 12.0 | |
| Special (Cognis) | Sulfate | | | | | | | | | |
| Texapon N 70 | Sodium Laureth | | 10.0 | | | | | | 12.0 | |
| (Cognis) | Sulfate | | | | | | | | | |
| Water, | Water (Aqua) | Ad | Ad | Ad | Ad | Ad | Ad | Ad | Ad | Ad |
| demineralized | | 100 | 100 | 100 | 100 | 100 | 100 | 100 | 100 | 100 |

### EXAMPLE 10

### Protection of Dyed Hair against UV-induced Colour Change (Comparative Study)

Procedure:

Blonde hair strands 10 cm long and 1.5 cm wide are washed twice with standard shampoo for 1 minute, rinsed for 1 minute with hand-hot water, combed and dried. They are then coated on both sides with a mixture of 3.0 g of Goldwell topchic 7-N medium blonde hair colouring and 3.0 g of developer lotion (6% H₂O₂) and massaged thoroughly for 0.5 minute. After a contact time of 30 minutes, the hair is rinsed under hand-hot water for 0.5 minute, washed with standard shampoo, rinsed for 1 minute with hand-hot water, combed and dried, and then the Lab values (0 h) are measured using a Minolta CR-300 chronometer.

The strands are combined in 3 groups and treated with 3.0 g of the active ingredient (see below) dissolved in ethanol, ethanol/water or water. After a contact time of one hour, the strands of hair are dried and then irradiated for 12 hours per side (front and back) with the Atlas Ci3000 Xenon Fade-Ometer at 615 W/m² and 300-800 nm. The hair strands are then washed with standard shampoo, rinsed with hand-hot water, combed and dried, and the Lab values (12 h) are measured. Treatment is then carried out for a second time with the respective active ingredient solution as described above. After irradiation again for 12 hours per side, the hair strands are again washed, rinsed, combed and dried. Finally, the Lab values (24 h) are measured.

The following was used as the standard shampoo:

| Ingredients | INCI | % (w/w) |
|---|---|---|
| Pantacare PS 10 | Sodium Laureth Sulfate, Lauryl Glycoside | 20.0 |
| (Cognis) | | |
| Water | Water | 76.8 |
| Sodium Chloride | Sodium Chloride | 1.4 |
| Dragocid Liquid | Phenoxyethanol, Methyl-, Butyl-, Ethyl-, | 0.5 |
| (Symrise) | Isobutylparaben | |
| Citric Acid | Citric Acid | 1.3 |

Calculation of the total colour difference ΔE using the example of the 24 h Lab values:

ΔE=(ΔL²+Δa²+Δb²)^{1/2}

where ΔL=L (24 h)−L (0 h), Δa=a (24 h)−a (0 h) and Δb=b (24 h)−b (0 h)

Calculation of the percentage colour change is carried out by relating the mean value (MV) of ΔE of the hair strands treated with the active ingredient to the mean value of ΔE of the control (=untreated hair strands):

Colour change [%]=(MV ΔE_{active ingredient}/MV ΔE_{control})×100

The colour-protecting activity can accordingly be calculated as follows:

Colour protection [%]=100−colour change [%]

Active Ingredients Used/Results:

In Tables 2 and 3 below

1=untreated (=control)

2=grape seed extract (Symrise), 0.1 wt. %

3=Rosmarin HD Stabiloton WS (rape), 1.0 wt. %

4=Neo-Extrapon Bio-Rooibos (Symrise), 1.0 wt. %

TABLE 2
Total colour difference ΔE values after 24 hours' exposure to UV

| Active | | | | | | | | | | | | | MV | SD | |
|---|---|---|---|---|---|---|---|---|---|---|---|---|---|---|---|
| ingredient | Total colour difference ΔE (24 h) | | | | | | | | | | | | ΔE | ΔE | p^{a} |
| 1 | 7.37 | 6.13 | 7.59 | 7.80 | 5.93 | 6.48 | 6.93 | 7.52 | 5.65 | 6.85 | 8.07 | 6.72 | 6.92 | 0.77 | — |
| 2^{b} | 5.64 | 6.75 | 5.12 | 4.57 | 6.37 | 3.77 | 8.12 | 5.40 | 6.26 | 5.16 | 6.09 | 6.37 | 5.80 | 1.12 | 0.010 |
| 3^{c} | 5.28 | 6.83 | 6.68 | 6.77 | 5.17 | 4.26 | 4.89 | 6.21 | 4.41 | 5.58 | 5.55 | 6.86 | 5.71 | 0.95 | 0.002 |
| 4^{d} | 5.13 | 5.01 | 5.89 | 6.57 | 4.53 | 4.67 | 5.40 | 5.52 | 5.77 | 6.89 | 3.49 | 3.69 | 5.21 | 1.03 | 0.0001 |

SD ΔE: Standard deviation of the ΔE values
^{a}test for significance compared with untreated control, Student's test (Excel, unpaired samples of equal variances). A significant activity is present at p < 0.05.
^{b}carrier-free extract, characterised by a total polyphenol content of 75.7% (Folin-Denis method, according to Example 13)
^{c}carrier-free extract, characterised by a content of 4.9% carnosolic acid and 0.8% carnosol (HPLC)
^{d}carrier material maltodextrin; 1.0 wt. % Neo-Extrapon Bio-Rooibos (Symrise), characterised by an aspalathin content of 0.01% (HPLC), contains 5 wt. % rooibos extract (aqueous extract prepared from fermented red bush)

Neo Extrapon Bio Rooibos consists of 95% maltodextrin and 5% rooibos extract. The content of aspalathin is 0.01%. Accordingly, 0.2% aspalathin is present in the pure, carrier-free rooibos extract.

The results from Table 2 clearly show that 1.0 wt. % Neo-Extrapon Bio-Rooibos, corresponding to 0.05% rooibos extract prepared by water extraction from fermented rooibos, exhibits significant colour-preserving activity.

TABLE 3
Colour protection after 24 hours' exposure

| Active | MV ΔE | SD ΔE | Colour change | Colour protection |
|---|---|---|---|---|
| ingredient | (24 h) | (24 h) | [%]^{a} | [%]^{a} |
| 1 | 6.92 | 0.77 | 100 | 0 |
| 2^{b} | 5.80 | 1.12 | 83.8 | 16.2 |
| 3^{c} | 5.71 | 0.95 | 82.5 | 17.5 |
| 4^{d} | 5.21 | 1.03 | 75.3 | 24.7 |

SD ΔE: standard deviation of the ΔE values
^{a}based on control (=untreated hair strands)
^{b}carrier-free extract, characterised by a total polyphenol content of 75.7% (Folin-Denis method, according to Example 13)
^{c}carrier-free extract, characterised by a content of 4.9% carnosolic acid and 0.8% carnosol (HPLC)
^{d}carrier material maltodextrin; 1.0 wt. % Neo-Extrapon Bio-Rooibos (Symrise), characterised by an aspalathin content of 0.01% (HPLC), contains 5 wt. % rooibos extract (aqueous extract prepared from fermented red bush).

The results from Table 3 clearly show that 1.0 wt. % Neo-Extrapon Bio-Rooibos containing 0.05% roobis extract prepared by water extraction from fermented rooibos exhibits very good colour-preserving activity. Based on the natural extract content (natural extract=carrier-free dry extract), the colour-protecting action exhibited by the rosemary extract containing the phenolic diterpenes carnosolic acid and carnosol is poorer by a factor of 28, and that of the grape seed extract is poorer by a factor of 3, compared with the rooibos extract.

### EXAMPLE 11

### Protection of Dyed Hair against UV-Induced Colour Change by Rooibos Extract Prepared by Water Extraction from Fermented Rooibos (Natural Extract)

The hair test from Example 10 was used as the test model. The procedure and calculations were carried out analogously to the description given therein. The fermented rooibos water extract was a carrier-free dry extract, characterised by an aspalathin content of 0.85% (HPLC).

The results of the test after 24 hours' exposure corresponded substantially to those of Example 10; the rooibos extract studied had very good colour-preserving activity.

### EXAMPLE 12

### Protection of Dyed Hair against UV-induced Colour Change by Rooibos Extract; Prepared by Water Extraction from “Green”, Unfermented Rooibos (Natural Extract)

The hair test from Example 10 was used as the test model. The procedure and calculations were carried out analogously to the description given therein. The “green”, unfermented rooibos water extract was a carrier-free dry extract, characterised by an aspalathin content of 8.4% (HPLC).

Results:

In Tables 4 and 5 below

1=untreated (=control)

2=grape seed extract (Symrise), 0.1 wt. %

3=“green”, unfermented rooibos extract, 0.05 wt. % (according to the invention)

TABLE 4
Total colour difference ΔE values

| | | | | |
|---|---|---|---|---|
| Active | | MV | SD | |
| ingredient | Total colour difference ΔE (12 h) | ΔE | ΔE | p^{a} |

a) ΔE values after 12 hours' exposure to UV (single treatment with active ingredient)

| | | | | | | | | | | | | | | | |
|---|---|---|---|---|---|---|---|---|---|---|---|---|---|---|---|
| 1 | 3.14 | 3.73 | 4.22 | 3.52 | 5.24 | 5.66 | 4.69 | 3.87 | 2.71 | 5.24 | 4.83 | 3.54 | 4.20 | 0.93 | — |
| 2^{b} | 5.17 | 2.91 | 2.17 | 2.31 | 2.62 | 2.78 | 3.67 | 3.26 | 2.85 | 3.51 | 4.18 | 5.34 | 3.40 | 1.04 | 0.059 |
| 3 | 3.18 | 2.33 | 3.52 | 4.46 | 3.04 | 3.37 | 4.38 | 3.95 | 3.33 | 2.66 | 2.84 | 2.62 | 3.31 | 0.68 | 0..014 |

b) ΔE values after 24 hours' exposure to UV (treatment twice with active ingredient)

| | | | | | | | | | | | | | | | |
|---|---|---|---|---|---|---|---|---|---|---|---|---|---|---|---|
| 1 | 5.68 | 6.29 | 6.41 | 5.36 | 8.10 | 7.83 | 6.93 | 7.52 | 5.65 | 6.85 | 8.0 | 6.72 | 6.78 | 0.95 | — |
| 2^{b} | 8.68 | 5.18 | 5.10 | 5.36 | 4.63 | 6.77 | 8.12 | 5.40 | 6.26 | 5.16 | 6.09 | 6.37 | 6.09 | 1.25 | 0.142 |
| 3 | 5.30 | 4.19 | 6.29 | 6.18 | 5.73 | 4.97 | 6.51 | 6.70 | 5.59 | 6.75 | 4.72 | 5.40 | 5.69 | 0.82 | 0.006 |

SD ΔE: Standard deviation of the ΔE values
^{a}test for significance compared with untreated control, Student's test (Excel, unpaired samples of equal variances). A significant activity is present at p < 0.05.
^{b}carrier-free extract, characterised by a total polyphenol content of 75.7% (Folin-Denis method, according to Example 13)

The results from Tables 4a and 4b clearly show that 0.05 wt. % “green”, unfermented rooibos water extract exhibits a significant colour-preserving activity both after 12 hours (single treatment with active ingredient) and after 24 hours (treatment twice with active ingredient).

TABLE 5
Colour protection after 12 and 24 hours' exposure

| | | | Colour | | | Colour |
|---|---|---|---|---|---|---|
| Active | | | protec- | | | protec- |
| ingre- | MV ΔE | SD ΔE | tion [%]^{a} | MV ΔE | SD ΔE | tion [%]^{a} |
| dient | (12 h) | (12 h) | (12 h) | (24 h) | (24 h) | (24 h) |
| 1 | 4.20 | 0.93 | 0 | 6.78 | 0.95 | 0 |
| 2^{b} | 3.40 | 1.04 | 19.1 | 6.09 | 1.25 | 10.2 |
| 3 | 3.31 | 0.68 | 21.2 | 5.69 | 0.82 | 16.1 |

SD ΔE: standard deviation of the ΔE values
^{a}based on control (=untreated hair strands)
^{b}carrier-free extract, characterised by a total polyphenol content of 75.7% (Folin-Denis method, according to Example 13)

### EXAMPLE 13

The results from Table 5 clearly show that 0.05 wt. % “green”, unfermented rooibos extract exhibits very good colour-preserving activity both after 12 and after 24 hours'exposure. Compared with the grape seed extract, its colour protection is better by a factor of 2.2 (12 h) or 3.2 (24 h).

### Determination of the Total Polyphenol Content

Procedure:

The determination of the total polyphenol content is carried out by photometry, the content of the extracts being determined as catechol equivalents by means of a catechol calibration curve.

For the preparation of the EDTA solution, 2.15 g of Titriplex III and 29.0 g of sodium hydroxide are dissolved with distilled water in a 1 litre measuring flask. A 0.5 to 1% solution of the extract to be determined in distilled water is prepared. For the calibration curve, catechol solutions of different concentrations in the range from 0.02 to 0.10 mg/ml in distilled water are prepared. 7.5 ml of distilled water, 1.5 ml of the EDTA solution, 1 ml of catechol solution, or 1 ml of extract solution, and 0.5 ml of Folin-Denis reagent are mixed and left to stand at room temperature for 30 minutes. In parallel, 1 ml of distilled water is prepared analogously as a blank sample. The absorption of the catechol and extract samples at 700 nm is then measured against the blank sample.

A calibration curve is prepared from the measured catechol absorption values. The total polyphenol content of the extract is calculated using the following equation:

Total polyphenol content [%]=(catechol equivalents from the calibration curve [mg/ml]/weighed amount of extract [mg/ml]×100)

Results:

TABLE 6

| | | Colour protection | |
|---|---|---|---|
| | Total polyphenol | [%] (24 h) | |
| Active ingredient | content [%] | (test concentration [%]) | |
| Grape seed extract | 75.7 | 13.2 | (0.1)^{a} |
| Neo-Extrapon Bio-Rooibos | 30.0^{c} | 24.7 | (1.0)^{b} |
| (Symrise)^{b} | | | |
| “Green” (unfermented) | 22.5 | 16.1 | (0.05) |
| rooibos water extract | | | |

^{a}mean value from Examples 10 and 12
^{b}carrier material maltodextrin; 1.0 wt. % Neo-Extrapon Bio-Rooibos (Symrise) containing 5 wt. % rooibos extract (aqueous extract prepared from fermented red bush), see Example 10
^{c}based on the natural extract (=carrier-free dry extract) corresponding to a total polyphenol content of 1.5% for the Neo-Extrapon Bio-Rooibos, see Example 12

The results from Table 6, in conjunction with the results from Examples 10 and 12, clearly show that there is no relationship between the total polyphenol content of an extract and its colour-protecting activity.

1. Use of an extract of fermented and/or unfermented rooibos leaves and/or stems to reduce or slow the loss of the natural or artificial colour of hair.

2. Use according to claim 1, wherein an extract of fermented rooibos leaves and/or stems is used.

3. Use according to claim 1 or 2, wherein the extract is an aqueous extract.

4. Use according to any one of the preceding claims, wherein the extract is used to reduce or slow the loss of the natural or artificial colour of hair as a result of light or treatment with a hair-cosmetic preparation.

5. Method of reducing or slowing the light-induced loss of the natural or artificial colour of hair, comprising the following step:
application of an extract of fermented and/or unfermented rooibos leaves and/or stems to the hair.

6. Method according to claim 5, wherein an extract of fermented rooibos leaves and/or stems is used.

7. Method according to claim 5 or 6, wherein the extract is an aqueous extract.

8. Method according to any one of claims 5 to 7, wherein the extract is used as a constituent of a hair-cosmetic preparation.

9. Method of reducing the hair-bleaching action or increasing the hair-colour-stabilising action of a hair-cosmetic preparation, wherein (a) an extract of fermented and/or unfermented rooibos leaves and/or stems is added to the preparation and/or (b) the extract of fermented and/or unfermented rooibos leaves and/or stems is applied to the hair before, during or after application of the preparation to the hair.
